(19) United States
(12) Patent Application Publication
(10) Pub. No.: US 2008/0123083 A1
(43) Pub. Date: May 29, 2008

(54) System and Method for Photoacoustic Guided Diffuse Optical Imaging

(75) Inventors: Xueding Wang, Canton, MI (US); Brian Fowlkes, Ann Arbor, MI (US); Paul Carson, Ann Arbor, MI (US); David Chamberland, Medford, OR (US)

Correspondence Address: BROOKS KUSHMAN P.C., 1000 TOWN CENTER, TWENTY-SECOND FLOOR, SOUTHFIELD, MI, 48075, US

(73) Assignee: THE REGENTS OF THE UNIVERSITY OF MICHIGAN, Ann Arbor, MI (US)

(21) Appl. No.: 11/947,321

(22) Filed: Nov. 29, 2007

Related U.S. Application Data
(60) Provisional application No. 60/861,590, filed on Nov. 29, 2006.

(51) Int. Cl. G01N 21/47; G01N 21/59; A61B 5/00

(52) U.S. Cl. 356/73; 600/437

(57) ABSTRACT

A system and method for photoacoustic guided diffuse optical imaging of a sample include at least one light source configured to deliver light to the sample, at least one ultrasonic transducer disposed adjacent to the sample for receiving photoacoustic signals generated due to optical absorption of the light by the sample, and at least one optical detector for receiving optical signals generated due to light scattered by the sample. A control system is provided in communication with the at least one light source, the ultrasonic transducer, and the optical detector for reconstructing photoacoustic images of the sample from the received photoacoustic signals and reconstructing optical images of the sample from the received optical signals. The priori anatomical information and spatially distributed optical parameters of biological tissues from the photoacoustic images employed in diffuse optical imaging may improve the accuracy of measurements and the reconstruction speed.

### CROSS-REFERENCE TO RELATED APPLICATIONS

This application claims the benefit of U.S. provisional application Ser. No. 60/861,590 filed Nov. 29, 2006 which is incorporated by reference herein.

### BACKGROUND OF THE INVENTION

1. Field of the Invention

This invention relates to photoacoustic guided diffuse optical imaging.

2. Background Art

Photoacoustic tomography (PAT) may be employed for imaging tissue structures and functional changes, and describing the optical energy deposition in biological tissues with both high spatial resolution and high sensitivity. PAT employs optical signals to generate ultrasonic waves. In PAT, a short-pulsed electromagnetic source—such as a tunable pulsed laser source, pulsed radio frequency (RF) source or pulsed lamp—is used to irradiate a biological sample. The photoacoustic (ultrasonic) waves excited by thermoelastic expansion are then measured around the sample by high sensitive detection devices, such as ultrasonic transducer(s) made from piezoelectric materials and optical transducer(s) based on interferometry. Photoacoustic images are reconstructed from detected photoacoustic signals generated due to the optical absorption in the sample through a reconstruction algorithm, where the intensity of photoacoustic signals is proportional to the optical energy deposition.

Optical signals, employed in PAT to generate ultrasonic waves in biological tissues, present high electromagnetic contrast between various tissues, and also enable highly sensitive detection and monitoring of tissue abnormalities. It has been shown that optical imaging is much more sensitive to detect early stage cancers than ultrasound imaging and X-ray computed tomography. The optical signals can present the molecular conformation of biological tissues and are related to significant physiologic parameters such as tissue oxygenation and hemoglobin concentration. Traditional optical imaging modalities suffer from low spatial resolution in imaging subsurface biological tissues due to the overwhelming scattering of light in tissues. In contrast, the spatial resolution of PAT is only diffraction-limited by the detected photoacoustic waves rather than by optical diffusion; consequently, the resolution of PAT is excellent (60 microns, adjustable with the bandwidth of detected photoacoustic signals). Besides the combination of high electromagnetic contrast and high ultrasonic resolution, the advantages of PAT also include good imaging depth, relatively low cost, non-invasive, and non-ionizing.

Recently, optical technologies based on diffusion light, including diffuse optical tomography (DOT), fluorescence optical diffusion tomography, and tomographic bioluminescence imaging have been employed widely in biomedical imaging to present tissue structural and functional information from tissue level to molecular and cellular levels. In DOT, light in the ultraviolet, visible or near-infrared (NIR) region is delivered to a biological sample. The diffusely reflected or transmitted light from the sample is measured and then used to probe the absorption and scattering properties of biological tissues. DOT is now available that allows users to obtain cross-sectional and volumetric views of various body parts. Currently, the main application sites are the brain, breast, limb, and joint.

DOT has a very good sensitivity and specificity in cancer detection and diagnosis based on the excellent optical contrast. Functional imaging with DOT offers several tissue parameters to differentiate tumors from normal background tissues, including blood volume, blood oxygenation, tissue light scattering, and water concentration. While DOT has the potential to improve tumor detection and diagnosis, its relatively low resolution makes it unsuitable for morphological diagnosis. Due to the high scattering of light in biological tissues, the edge and foci of imaged tumors are drastically blurred. Moreover, in DOT the recovery of spatially distributed optical parameters from measured signals requires the solving of an inverse problem, nonlinear in the optical parameters, and known to be severely underdetermined and ill-posed. As a result, accurate quantification and localization of optical parameters, including both morphological and physiological changes, in biological tissues are difficult to be achieved.

More recently, there has been great interest in adapting the methodologies of DOT to fluorescent imaging and bioluminescence imaging, as both of them enable the visualization of genetic expression and physiological processes at the molecular level in living tissues. The advantage of fluorescence imaging and bioluminescence imaging is that they present the high sensitivity and specificity of fluorescent dye tagging and reporter gene tagging. Although the spatial resolution is limited when compared with other imaging modalities, DOT provides access to a variety of physiological parameters and molecular changes that otherwise are not accessible, including sub-second imaging of hemodynamics and other fast-changing processes. Furthermore, DOT can be realized in compact, portable instrumentation that allows for bedside monitoring at relatively low cost.

Attaining the potential to provide three-dimensional quantified images of novel fluorescent and bioluminescent taggings in intact tissues, DOT has been employed to advance the emerging field of optical molecular imaging. Recently, the development of DOT imaging systems has enabled the application of fluorescence molecular tomography (FMT), a technique that resolves molecular signatures in deep tissues using fluorescent probes or markers. The performance of FMT in vivo in three-dimensional imaging of enzymatic activity in deep-seated tumors has been demonstrated in small animals. Bioluminescence tomography (BLT), as an emerging imaging technique, is a major frontier of bioluminescence imaging. Employing DOT, the molecular luminescence from luciferase is used to reconstruct its spatial distribution and to visualize local functional, physiology, or genetic activation within tissues.

Optical imaging requires that an array of sources and detectors be distributed directly or coupled through optical fibers on a boundary surface of the sample. Sinusoidally modulated continuous-wave or pulsed excitation light is launched into the biological tissues, where it undergoes multiple scattering and absorption before exiting. One can use the measured intensity and phase (or delay) information to reconstruct 3D maps of a tissue's optical properties by optimizing a fit to diffusion model computations. As a result of the nonlinear dependence of the diffusion equation photon flux on the unknown parameters and the inherently 3D nature of photon scattering, this inverse problem is computationally intensive and must be solved in an iterative means. The estimation of each of the unknown images from the corresponding observations is normally an ill-posed, typically underdetermined, inverse problem.

### BRIEF DESCRIPTION OF THE DRAWINGS

FIG. 1 is a schematic diagram of a photoacoustic guided diffuse optical imaging system according to one aspect of the present invention.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for teaching one skilled in the art to variously employ the present invention.

In accordance with the present invention, a photoacoustic guided diffuse optical imaging system and method for medical imaging, monitoring and diagnosis are provided which may employ both photoacoustic tomography (PAT) and diffuse optical imaging. The photoacoustic components in this system can provide morphological properties and optical information of subsurface biological tissues with high spatial resolution and high optical contrast. This priori anatomical information and spatially distributed optical parameters of biological tissues visualized by PAT may be further employed in diffuse optical imaging to significantly improve the accuracy of measurements and the reconstruction speed. With the priori tissue anatomical information provided by PAT, local functional parameters in biological samples (e.g., blood volume, blood oxygenation, tissue scattering and water concentration in cancerous tumors) can potentially be quantified with much improved specificity. Quantitative and three-dimensional imaging of fluorescent and bioluminescent sources or contrast agents in high scattering biological samples can also be advanced with much better accuracy and higher spatial resolution.

The hybrid imaging system according to the present invention may also enable co-registration of photoacoustic and optical diffuse images of the tissue sample under study. Co-registered images provide high spatial resolution and high tissue contrast which are enabled by PAT, and high sensitivity and high contrast in functional imaging at tissue, cellular or molecular level which are inherited from diffuse optical imaging. The system and method according to the present invention retain the contrast and sensitivity advantages of diffuse optical imaging modalities while enhancing their spatial resolution, accuracy, stability and specificity, which greatly broaden and strengthen the potential application of current optical imaging modalities in medicine and biology.

The photoacoustic guided diffuse optical imaging system according to the present invention may generate photoacoustic images and optical images of the same sample. Photoacoustic and diffuse optical images can be acquired sequentially or simultaneously. Photoacoustic images present tissue structures clearly based on the high optical contrast (e.g., the border and foci of a tumor or a multilayer skin structure). Because the spatial resolution of PAT is limited mainly by the bandwidth of detected photoacoustic signals rather than by optical diffusion as in DOT, PAT is able to describe point-by-point tissue morphological structures with an excellent spatial resolution (e.g., 100 microns in small animal brain imaging). The contrast in photoacoustic images reveals the distribution of optical energy deposition in various tissues, which is a product of the local light energy fluence and the local optical absorption coefficient. When the light fluence is nearly homogeneous in the imaging space, or the distribution of light fluence in the sample can be measured or simulated, PAT can present a distribution of relative optical absorption. The relative optical absorption is an important parameter in imaging and diagnosis of tissue abnormalities and functional activities. From photoacoustic outcomes, the attenuation coefficients of various biological tissues, which is another important diagnostic optical parameter, can also be measured. The 3D imaging and quantification of these optical parameters may contribute to the image reconstruction in diffuse optical imaging. For example, in the quantifying imaging of fluorescent or bioluminescent sources in a small animal tumor model, the effect of light attenuation in the tissue layers (e.g., fat, muscle and skin) covering the tumor can be removed when the attenuation coefficients and the thicknesses of these tissue layers can be measured through PAT.

Diffuse optical images can present tissue optical properties, including optical scattering coefficients and optical absorption coefficients, and tissue physiological and chemical parameters (e.g., blood oxygenation, blood volume and water concentration). When fluorescence or bioluminescence imaging is conducted, diffuse optical imaging is able to describe 3D distribution and changes of fluorescent or bioluminescent sources or contrast agents in subsurface tissues. Although the spatial resolution of optical imaging is poor due to the high scattering of light in tissues, the sensitivity of diffuse optical imaging modalities in functional measurement is excellent, higher than MRI, ultrasound and x-ray CT. Again, diffuse optical imaging of deep objects in tissues requires the application of advanced excitation-detection schemes and the use of tomographic reconstructions based on the diffusion theory combined with data acquired at different projections. The estimation of each of the unknown images from the corresponding observations is an ill-posed, typically underdetermined, inverse problem. This problem in diffuse optical imaging can be addressed by introduction of priori information, including both anatomical information and optical parameters of tissues obtained from PAT.

PAT guided diffuse optical imaging according to the present invention may provide three-dimensional quantified images of optical properties or fluorescent or bioluminescent sources or contrast agents provided within intact tissues with much improved accuracy than diffuse optical imaging alone. The priori anatomical information from three-dimensional photoacoustic images may contribute to optical imaging by reducing computational burden and improving accuracy and robustness. In comparison with other traditional imaging modalities (e.g., ultrasound, x-ray, CT, and MRI), PAT presents tissue anatomy based on the more direct measurements of tissue optical properties and, as a result, may lead to a better guidance for diffuse optical imaging that studies spatially distributed optical parameters in the same sample. In other words, because both PAT and diffuse optical imaging are based on tissue optical contrast and visualizing tissue optical properties, PAT outcomes, in comparison with the measurements from other imaging modalities, are more compatible when used in the guidance of diffuse optical imaging. Because the wavelength in PAT is tunable, optical parameters in tissues as functions of light spectrum can be analyzed before diffuse optical imaging, which may be especially important in guiding fluorescence or bioluminescence imaging where more than one light wavelength is involved.

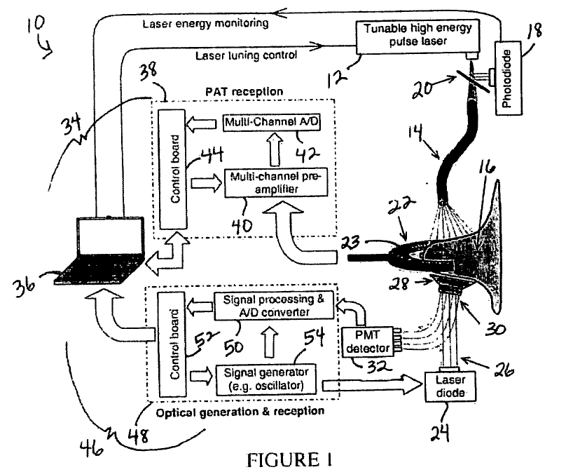

An imaging system according to the present invention may include laser delivery and wavelength tuning for PAT, photoacoustic signal generation and reception for PAT, reconstruction of photoacoustic images, light generation and delivery for diffuse optical imaging, detection and processing of forward-transmitted or diffusely-reflected optical signals, and reconstruction of diffuse optical images. FIG. 1 is a schematic diagram of a system 10 for photoacoustic guided diffuse optical imaging in accordance with the present invention.

The system 10 includes components for photoacoustic tomography and components for diffuse optical imaging which are integrated together into a hybrid system. According to one aspect of the present invention, at least one light source or laser 12, such as an optical parametric oscillator (OPO) laser system pumped by an Nd:YAG laser working at 532 nm (second-harmonic), may be used for photoacoustic imaging to provide pulses (e.g., ˜5 ns) which may have a tunable wavelength, such as ranging between 680 nm and 950 nm. Other spectrum regions can also be realized by choosing other tunable laser systems (e.g., Ti:Sapphire laser, dye laser, or OPO pumped by 355 nm Nd:YAG laser) or lamps. The light source 12 for PAT according to the present invention may be any device that can provide short light pulses with high energy, short linewidth, and tunable wavelength, and other configurations are also fully contemplated. Short light pulse duration (e.g., 5 ns) is typically necessary for efficient generation of photoacoustic signals. Through free space or an optical fiber bundle 14, laser light may be delivered to a sample 16 (e.g., human breast) with an input energy density below the ANSI safety limit. The delivered laser energy can be monitored by an optical sensor (e.g. photodiode) 18, which may be facilitated by a beam splitter 20.

Pulsed light from the light source 12 may induce photoacoustic signals in an imaged sample 16 that may be detected by a transducer 22, such as a high-sensitivity, wide-bandwidth ultrasonic transducer, to generate 2D or 3D photoacoustic tomographic images of the sample 16. The spatially distributed optical energy in the sample 16 generates proportionate photoacoustic waves due to the optical absorption of biological tissues (i.e., optical energy deposition). The signal between the sample 16 and the transducer 22 may be coupled with any suitable ultrasound coupling material such as, but not limited to, water, mineral oil and ultrasound coupling gel. A focused ultrasound transducer (or a transducer array) may be employed for signal receiving and images generated directly as in traditional ultrasonography, or photoacoustic signals may also be received with non-focused transducer(s) and images reconstructed through a reconstruction algorithm. Other high sensitive ultrasound detection devices, such as an optical transducer based on interferometry, can be used instead of transducer 22. When the PAT light source 12 is tunable, photoacoustic images are able to be obtained corresponding to different wavelengths, thereby achieving functional spectroscopic photoacoustic tomography of the sample 16.

Transducer 22 can be any ultrasound detection device, e.g. single element transducers, 1D or 2D transducer arrays, optical transducers, transducers of commercial ultrasound machines, and others. Transducer 22 may employ a 1D array 23 that is able to achieve 2D imaging of the cross section in the sample 16 surrounded by the array 23 with a single laser pulse. The imaging of a 3D volume in the sample 16 may be realized by scanning the array 23 along its axis. In order to achieve 3D photoacoustic imaging at one wavelength with a single laser pulse, a 2D transducer array 23 could instead be employed for signal detection. The photoacoustic signals can be scanned along any surfaces around the sample 16.

The parameters of ultrasonic transducer 22 include element shape, element number, array geometry, array central frequency, detection bandwidth, sensitivity, and others. The design of the transducer 22 in the system 10 according to the present invention may be determined by the imaging purpose and the sample 16, including the shape of studied sample 16, the expected spatial resolution and sensitivity, the imaging depth, and others. For example, for PAT of human breast, a 2D semispherical transducer array 23 can be applied, which can realize a high speed or even real-time photoacoustic tomography of the breast. Transducer elements, which may be distributed evenly along a semispherical surface around the breast, can collect photoacoustic signals simultaneously through all the directions with a 2, solid angle. Instead of a 2D transducer array 23, a 1D semicircular transducer array 23 may also be utilized for breast imaging. According to one non-limiting aspect of the present invention, the design of array 23 may be: central frequency of 2 MHZ, bandwidth of 100%, pitch size of 0.75 mm (1λ at central frequency), array size of 12 cm in diameter, number of elements of 256, and array elevation height of 0.75 mm. This transducer 22 may realize 2D cross-sectional imaging of a breast with a spatial resolution of 0.75 mm and a fast imaging speed (only limited by the signal-noise ratio). In order to realize 3D imaging of a breast with the 2D semispherical transducer array 23, this transducer 22 may need to be scanned around the breast. Of course, other configurations (e.g., ring-shaped, spherical, etc.) of the transducer 22 and its array 23 are also fully contemplated.

Ultrasonic transducer 22 may also be used to realize conventional gray scale ultrasound imaging and Doppler ultrasound of the sample 16 by using the ultrasonic transducer 22 as both a transmitter and receiver of ultrasound signals and appropriate existing signal processing circuitry. Furthermore, ultrasound images and image volumes may be fused with or registered to images and image volumes such as PET, CT, and MRI, and with other ultrasound modes that may have the desired contrast or freedom from noise or artifacts to serve as a guide for optical reconstructions.

For diffuse optical imaging, a laser diode(s) may be employed as a continuous-wave (CW) light source 24. The light source 24 for diffuse optical imaging according to the present invention may be any device that can provide CW or pulsed light, such as, but not limited to, a diode laser, dye lasers, and arc lamps. PAT and DOT may also share the same light source, for example, but not limited to, pulsed light from a dye laser. In this case, only one light source may be employed in the hybrid imaging system 10 according to the present invention. When pulsed light is delivered to the sample 16, part of the energy will be absorbed by the tissues that generate photoacoustic signals, while the other part of the energy will be scattered from the sample 16, enabling diffuse optical imaging of the same sample 16 such that diffuse optical images and photoacoustic images may be realized simultaneously.

The wavelength spectrum of the light pulses for PAT and diffuse optical imaging may be selected according to the imaging purpose, specifically the optical properties, functional parameters, and fluorescent or bioluminescent sources/contrast agents within the sample 16 to be studied. The studied spectral region may range from ultraviolet to infrared (300 nm to 1850 nm), but is not limited to any specific range.

The light from the light source 24 may be delivered through source fibers 26 may be directed via a probe 28 to a surface of the sample 16, for example, but not limited to, a human breast. Once the light photons enter tissues, the trajectories of the photons are changed quickly due to the overwhelming scattering property of tissues. The scattered photons, except those absorbed by tissues, exit the sample 16 through all directions. The light energy may be delivered to the sample 16 through any methods, such as free space beam path and optical fiber(s). As an example, the diffusely reflected light in FIG. 1 may be collected by detection fibers 30 which may be distributed on the sample 16 surface via the probe 28. The number of source fibers 26 and detection fibers 30 as well as their spatial distributions on the surface of the DOT probe 28 are parameters that may be selected to determine the imaging quality and accuracy.

In the system 10 according to the present invention, light delivered to the sample 16 can be measured in a forward mode (transmittance), a backward mode (diffuse reflectance), or a side mode by an optical detector 32 such as photon multiplier tubes (PMT) to achieve diffuse optical imaging of the sample 16. The detection of transmitted or diffusely reflected light for diffuse optical imaging can also be realized through CCD, photodiode, avalanche photodiode (APD), or any other light detection devices. If multiple wavelengths are applied, spectroscopic optical imaging of the same sample 16 is achievable.

With reference again to FIG. 1, the photoacoustic signals detected by the transducer 22 may be communicated to a PAT control system 34, which may include a processor/controller, such as a computer 36, and PAT reception circuitry 38. Reception circuitry 38 may include an amplifier 40 (e.g., multi-channel preamplifier with, for example, 64, 128, or 256 channels), an A/D converter 42 (e.g., multi-channel A/D converter with, for example, 64, 128, or 256 channels), and a control board 44 in communication with the computer 36, the amplifier 40, and the A/D converter 42. As such, the photoacoustic signals detected by the transducer 22 may be amplified, digitized, and then sent to the computer 36. The control system 34 may also receive the triggers from the laser 12 and record the laser pulse energy detected by the photodiode 18. At the same time, the control system 34 may also control the tuning of the wavelength of the laser 12 and the scanning of the transducer 22 when necessary. Photoacoustic tomographic images may be reconstructed from detected signals through a reconstruction algorithm. It is understood that the control system 34 shown in FIG. 1 is only an example, and that other systems with similar functions may also be employed in the system 10 according to the present invention for control and signal receiving.

For diffuse optical imaging, the received optical signals containing phase, intensity and spatial information may be sent from detector 32 to an optical generation/reception control system 46 including optical generation/reception circuitry 48 and the computer 36. The received optical signals may be digitized by an A/D converter 50 and then sent to the computer 36, such as via a control board 52, to generate optical images. The signal processing circuitry 48 may also include an amplifier, filter, and/or mixer, as well as other devices. The reconstruction of optical images, including both absorption and scattering images, can be realized through an algorithm based on diffusion theory. For optical signal generation, the computer 36 and control board 50 may direct a signal generator 52 (e.g., oscillator) in communication with the laser diode 24 for modulating the output thereof. It is understood that the control system 46 shown in FIG. 1 is only an example, and that other systems with similar functions may also be employed in the system 10 according to the present invention for control and signal receiving. Of course, it is also understood that control systems 34 and 46 may also be embodied as a single, integrated unit.

When fluorescent contrast agents are employed in biological tissues for fluorescence imaging, fluorescent light that has a spectral shift from the incident light wavelength may be collected. In order to avoid potential photo bleaching of the contrast agent in tissues caused by the strong light pulses for PAT, photoacoustic imaging may be applied after fluorescent imaging. For bioluminescence imaging, no incident light is needed and the optical imaging system will collect only the diffusely scattering light emitted from the spatially distributed bioluminescent sources in tissues. In general, multiple different types of contrast agents could be used on the same sample 16 over a period of time which could enhance the data for the specific tissue involved, with the added benefit of facilitated image registration due to the integrated nature of the system 10.

Some optical contrast agents (e.g., gold colloids and other metallic colloids, quantum dots, carbon nanoparticles, and some biological dyes) present both fluorescent and strong optical absorption. For example, the dynamic distribution of a fluorescent contrast agent (targeting or non-targeting) in biological tissues can be imaged by both PAT and fluorescent imaging. The geometric information and tissue optical properties provided by photoacoustic images will contribute to the reconstruction of fluorescent images. As another example, gold nanoparticles (e.g., rods, cages, spheres, etc.) present very strong optical scattering and optical absorption, and thus may be used as a contrast agent for PAT and DOT in system 10. Gold has been used for therapeutic pharmaceutical use in inflammatory arthritis, specifically rheumatoid arthritis. Gold nanoparticles may also be conjugated to current existing anti-rheumatic drugs, anti-tumor necrosis factor drugs, anti-CD20 drugs, or others, thus producing a bioactive contrast agent. The use of gold, such as in a nanoparticle form, whether or not conjugated with drugs, may be beneficial for use with the system 10 due to its contrast and therapeutic effects. Through this dual-modality imaging system and method according to the present invention, a high resolution, accurate, quantitative evaluation of an optical contrast agent and associated tissue morphological and physiological parameters can be achieved, which cannot be realized through traditional diffuse optical imaging modalities or PAT alone.

Again, PAT visualizes tissue structures and functional changes based on the optical contrast. Tissue optical parameters that can potentially be measured from photoacoustic images include, but are not limited to, tissue optical absorption coefficients and tissue attenuation coefficients. The morphological and optical information of the imaged sample can then be drawn from photoacoustic outcomes. This information may then be employed to guide the inverse problem in diffuse optical imaging to calculate and quantify the optical parameters to be studied (e.g., tumor blood oxygenation and blood volume, and distribution and change of fluorescent or bioluminescent sources). The reconstruction of optical images can be realized through a certain algorithm based on diffusion theory.

In accordance with the present invention, the sample 16 to be studied using the system 10 can be any sample, such as a living organism, animals, or humans. The system and method according to the present invention may be used on any part of the human body and adaptations may be made when different organs need to be imaged such as, but not limited to, the breast, brain, skin, and joint. Also, the system and method according to the present invention could be incorporated into invasive probes such as those used for endoscopy including, but not limited to, colonoscopy, esophogastroduodenoscopy, bronchoscopy, laryngoscopy, and laparoscopy. The system and method described herein can also be used for other biomedical imaging, including those conducted on animals. The performance of the system may be invasive or non-invasive, that is, while the skin and other tissues covering the organism are intact. In addition, the system and method according to the present invention may be suitable for industrial or manufacturing purposes such as, but not limited to, fluid analysis.

The computer 36 in the system 10 according to the present invention may refer to any suitable device operable to execute instructions and manipulate data, for example, a personal computer, work station, network computer, personal digital assistant, one or more microprocessors within these or other devices, or any other suitable processing device.

The reception of photoacoustic signals and the transmission and reception of optical signals can be realized with any proper designs of circuitry and any scanning geometry. The circuitry 38, 48 may perform as an interface between the computer 36 and the transducer 22, laser 12, photodiode 18, PMT detector 32, laser diode 24, and other devices. “Interface” may refer to any suitable structure of a device operable to receive signal input, send control output, perform suitable processing of the input or output or both, or any combination of the preceding, and may comprise one or more ports, conversion software, or both. A component of a reception system 34, 46 may comprise any suitable interface, logic, processor, memory, or any combination of the preceding.

According to the present invention, the reconstruction method used in the system 10 to generate photoacoustic signals can be any basic or advanced algorithms, such as simple back-projection, filtered back-projection, and other modified back-projection methods. The reconstruction of photoacoustic tomographic images may be performed in both spatial domain and frequency domain. The reconstruction used in this system 10 to generate optical images can be any basic or advanced algorithms based on diffusing theory or other theories, and the reconstruction of optical images can be performed in either spatial domain or frequency domain. Before or after the generation of photoacoustic and optical images, any signal processing methods can be applied to improve the imaging quality. Images may be displayed on the computer 36 or another display.

In further accordance with the present invention, the system can also be adapted to realize microwave imaging guided by microwave thermoacoustic imaging. By using a pulsed microwave source(s) instead of a pulsed laser source for photoacoustic imaging, thermoacoustic imaging can be realized which is also based on the thermoelastic expansion of tissues due to the absorption of short-pulse electromagnetic waves. In comparison with traditional microwave imaging, microwave induced thermoacoustic imaging has both high contrast and good spatial resolution. Therefore, the anatomical information and tissue properties measured by microwave induced thermoacoustic imaging may help improve microwave imaging by reducing computation burden and enhancing accuracy, robustness, specificity and spatial resolution.

The system and method according to the present invention are amenable for use in the diagnosis and therapeutic monitoring of inflammatory arthritis, specifically rheumatoid arthritis. In inflammatory arthritis, there is increased angiogenesis and, as an extension, increased localized hemoglobin which may be much better detected with the combination of PAT and DOT as in the present invention than with other modalities. While depth can be an issue for DOT, one of the most common areas affected by rheumatoid arthritis, the finger joint, requires only very superficial imaging. Furthermore, ultrasound may also be used in combination with PAT and DOT as a complimentary imaging modality for rheumatoid arthritis, specifically for evaluating synovitis and erosions of periarticular bone.

Still further, the system and method according to the present invention may be used for the detection and diagnosis of various diseases, such as scleroderma and variants such as eosinophilic fasciitis, lupus, Raynauds phenomenon or other conditions with vasospastic changes affecting the digital arteries, Buergers disease, vasculitis including temporal arteritis, and general vascular disease, specifically peripheral vascular disease. The hybrid imaging system and method according to the present invention may also be used for noninvasive, non-ionizing monitoring of drug therapy of diseases including, but not limited to, cancer and inflammatory arthritis.

The system and method according to the present invention present high spatial resolution and high tissue contrast enabled by photoacoustic imaging and high sensitivity and high specificity in functional imaging which is inherited from optical imaging. The information that can be revealed by the system 10 according to the present invention includes, but is not limited to, 3D quantified tissue morphological features based on tissue intrinsic or extrinsic optical properties, 3D quantified functional parameters in local tissues, and 3D quantified distributions of fluorescent or bioluminescent sources in tissues. This system and method may contribute to improved detection and diagnosis of cancers with diffuse optical tomography (DOT), and may also help to localize and quantify fluorescent and bioluminescent sources in fluorescence imaging and bioluminescence imaging.

The photoacoustic guided diffuse optical imaging system and method according to the present invention may extract complementary information of biological tissues that cannot be realized by current existing imaging modalities. First, the system 10 may describe tissue structures and properties with both high ultrasound resolution and good optical contrast. With the priori tissue anatomical information provided by PAT, local optical properties and functional parameters in biological samples 16 may be quantified with much improved accuracy. With this system 10, quantitative and three-dimensional imaging of fluorescent and bioluminescent sources in high scattering biological samples may also be achieved with much better accuracy and higher spatial resolution. Second, in comparison with other available imaging modalities (e.g., ultrasound, x-ray, CT, and MRI), PAT presents tissue anatomy based on the more direct measurement of tissue optical properties and, as a result, may lead to a better guidance for diffuse optical imaging that studies spatially distributed optical parameters in the same sample 16. Third, with the design described above, different segments in this system 10 can be efficiently utilized. For example, the laser 12 may perform as the source for both PAT and diffuse optical imaging. Moreover, the imaging of a sample 16 by the integrated, dual-modality system according to the present invention may save time for image acquisition. Furthermore, the photoacoustic and optical imaging results of the same sample 16 may be combined together through image registration and used to provide comprehensive diagnostic information.

The system and method according to the present invention utilize the features of each imaging modality, many of which are complimentary and obviate the need for independent, fully-functioning systems, to create an enhanced hybrid image. The combination of two imaging technologies in one system as described herein enables comprehensive imaging functions and features that cannot be realized by existing imaging modalities. Second, this combination is not a simple group of two imaging systems, but instead a systematic integration of them. The imaging modalities realized by the system and method according to the present invention can benefit from each other, and the different segments in the system can be most efficiently utilized. Moreover, the imaging of a sample by an integrated dual-modality system as described herein can not only save the time and money for data acquisition in comparison with performing different modalities separately, but also make data registration more convenient and location more reproducible as all data may be acquired in real time.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A system for photoacoustic guided diffuse optical imaging of a sample, the system comprising:
at least one light source configured to deliver light to the sample;
at least one ultrasonic transducer disposed adjacent to the sample for receiving photoacoustic signals generated due to optical absorption of the light by the sample;
at least one optical detector for receiving optical signals generated due to light scattered by the sample; and
a control system in communication with the light source, the ultrasonic transducer, and the optical detector for reconstructing photoacoustic images of the sample from the received photoacoustic signals and reconstructing optical images of the sample from the received optical signals.

2. The system according to claim 1, wherein the at least one light source includes a pulsed light source.

3. The system according to claim 1, wherein the at least one light source includes a first light source and a second light source.

4. The system according to claim 3, wherein the second light source includes a continuous-wave light source.

5. The system according to claim 3, wherein the second light source includes a pulsed light source.

6. The system according to claim 3, further comprising a probe for delivering light from the second light source to the sample.

7. The system according to claim 6, wherein the probe includes source fibers in communication with the second light source, and detection fibers in communication with the optical detector.

8. The system according to claim 3, wherein the control system includes a signal generator for modulating an output of the second light source.

9. The system according to claim 1, wherein the at least one light source includes a microwave source for enabling thermoacoustic imaging of the sample.

10. The system according to claim 1, further comprising an optical sensor in communication with the control system for monitoring an energy of the delivered light.

11. The system according to claim 1, wherein the control system receives a firing trigger from the at least one light source.

12. The system according to claim 1, wherein the control system controls tuning a wavelength of the at least one light source.

13. The system according claim 1, wherein the ultrasonic transducer includes a semispherical array.

14. The system according to claim 1, wherein the control system is configured to combine the photoacoustic images and the optical images of the sample through image registration.

15. The system according to claim 1, wherein the sample includes a human breast.

16. The system according to claim 1, further comprising a contrast agent including gold nanoparticles provided within the sample.

17. The system according to claim 1, further comprising a fluorescent or bioluminescent contrast agent provided within the sample.

18. A method for photoacoustic guided diffuse optical imaging of a sample, the method comprising;
providing at least one light source for delivering light to the sample;
receiving photoacoustic signals generated due to optical absorption of the light by the sample with at least one ultrasonic transducer;
receiving optical signals generated due to light scattered by the sample with at least one optical detector; and
reconstructing photoacoustic images from the received photoacoustic signals and reconstructing optical images from the received optical signals.

19. The method according to claim 18, wherein providing the at least one light source includes providing a first light source and a second light source.

20. The method according to claim 19, further comprising modulating an output of the second light source via a signal generator.

21. The method according to claim 18, further comprising monitoring an energy of the delivered light via an optical sensor.

22. The method according to claim 18, further comprising receiving a firing trigger from the at least one light source.

23. The method according to claim 18, further comprising tuning a wavelength of the at least one light source.

24. The method according claim 18, wherein the ultrasonic transducer includes a semispherical array.

25. The method according to claim 18, further comprising combining the photoacoustic images and the optical images of the sample through image registration.

26. The method according to claim 18, wherein the sample includes a human breast.

27. The method according to claim 18, further comprising providing a contrast agent including gold nanoparticles within the sample.

28. The method according to claim 18, further comprising providing a fluorescent or bioluminescent contrast agent within the sample.

29. A system for photoacoustic guided diffuse optical imaging, the system comprising:
a first light source configured to deliver light pulses to a sample;
an ultrasonic transducer disposed adjacent to the sample for receiving photoacoustic signals generated due to optical absorption of the light pulses from the first light source by the sample;
a second light source configured to deliver light to the sample;
an optical detector for receiving optical signals generated due to light scattered by the sample upon delivery of light by the second light source; and
a control system in communication with the first light source and the second light source, in communication with the ultrasonic transducer for reconstructing photoacoustic images from the received photoacoustic signals, and in communication with the optical detector for reconstructing optical images from the received optical signals, wherein the control system is configured to combine the photoacoustic images and the optical images of the sample through image registration.
